(19) United States
(12) Patent Application Publication
(10) Pub. No.: US 2005/0080448 A1
(43) Pub. Date: Apr. 14, 2005

(54) Medical retrieval devices and related methods of use

(76) Inventors: Jason W. Kear, Bloomington, IN (US); Eric Cheng, Bloomington, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/942,087

(22) Filed: Sep. 16, 2004

Related U.S. Application Data
(60) Provisional application No. 60/503,821, filed on Sep. 18, 2003.

(51) Int. Cl. A61M 29/00

(52) U.S. Cl. 606/200

(57) ABSTRACT

Devices and related methods of use are provided for the entrapment and removal of various unwanted objects present within the body's anatomical lumens. In an embodiment of the present disclosure, the device includes a handle and a core element having a substantially linear first section and a second section capable of transitioning between an expanded configuration and a substantially linear configuration.

### CROSS-REFERENCE TO RELATED APPLICATION

This patent application claims the benefits of priority under 35 U.S.C. §§ 119, 120 to U.S. Provisional Patent Application Ser. No. 60/503,821, entitled MEDICAL RETRIEVAL DEVICE, filed on Sep. 18, 2003, the entirety of which is incorporated herein by reference.

### DESCRIPTION OF THE INVENTION

1. Field of the Invention

Embodiments of this invention relate generally to the field of medical devices and procedures. More particularly, embodiments of the instant invention relate to medical devices and procedures for immobilizing, manipulating, and/or removing objects, such as a stone, from within anatomical lumens.

2. Background of the Invention

Concretions, such as calculi, can develop in any part of the body. However, calculi typically develop in the passages of the biliary and urinary tracts, such as in the kidneys, pancreas, and gallbladder. Calculi are usually composed of salts of inorganic or organic acids, or other materials such as cholesterol.

Minimally invasive medical procedures have been utilized to clear blockages and remove objects from the anatomical lumens. Lithotripsy and ureteroscopy, for example, are techniques used to treat urinary calculi, e.g., kidney stones, in the ureter of patients. Lithotripsy is a medical procedure that uses energy in various forms, such as acoustic or electrical shock waves, pneumatic or hydraulic pulsation, or laser beams, to break up biological concretions such as urinary calculi. The force of the energy, when applied either extracorporeally or intracorporeally, usually in focused and continuous or successive bursts, comminutes a concretion into fragments of various sizes that may be extracted from the body or allowed to pass through urination.

Some retrieval devices are designed to extract biological concretions without the necessity of major open surgery. For example, a retrieval device may feature a basket for entraining biological stones during a lithotripsy procedure and safely removing them from the body. The basket may be deformable, collapsing upon retraction into a tubular sheath and expanding upon extending the basket beyond the distal end of the sheath.

Such devices, however, may suffer from at least two limitations. First of all, they may be susceptible to laser energy used in lithotripsy procedures. The laser energy not only fragments the concretion to be removed but may also weaken the strength of the devices. Furthermore, such devices make use of actuating mechanisms that may increase the overall size of the device and undesirably reduce the maneuverability and non-invasiveness of these devices.

The present disclosure provides medical retrieval devices and methods of using the same that address some or all of the aforementioned shortcomings of the existing devices.

### SUMMARY OF THE INVENTION

Accordingly, it is an object of at least certain embodiments of the present invention to provide a device that is more laser resistant than the currently known devices. Another object of at least certain embodiments of the present invention is to provide an alternative mechanism for actuating the currently known devices.

Embodiments of the invention provide a medical device and procedure for engaging material within a body. Methods in accordance with various embodiments of the present invention involve using the apparatus to grasp, move, remove, loosen, occlude, release, and/or sweep material in a body lumen of a patient.

In one aspect, the invention may be directed to a medical device having a core element composed of a laser resistant material. The core element may include a first section that extends substantially longitudinally and a second section that may be wound to form a helical coil. In one embodiment according to the invention, the core element may also include a lumen that extends longitudinally from at least a portion of the first section to at least a portion of the second section. The core element may further include an imperforate wall that defines the lumen. Alternatively, the wall may be perforated. The thickness of the wall may be maximized to enhance the laser resistance of the core element, while simultaneously maintaining a lumen with a diameter sufficient to receive an elongated member as discussed below.

Embodiments according to this aspect of the invention can include the following features. For example, the laser resistant material may be a shape memory material such as a polymeric material. The polymeric material may preferably be a light color to reflect most of the laser energy used during a lithotripsy procedure with minimal damage to the core element at normal laser operating levels.

Optionally, the medical device may further include an elongated member axially positioned and slideably moveable with the lumen of the core element. The second section of the core element may spontaneously form a helical coil in the shape memory material. The helical coil may adapt a substantially linear configuration when the elongated member is pushed along a section of the lumen that extends through the second section of the core element.

In one embodiment, the helical coil of the core element may taper in diameter from a larger diameter at the proximal end of the second section to a small diameter at the distal end of the second section, for example. Alternatively, the helical coil may have a larger diameter at the distal end and a smaller diameter at the proximal end of the second section. The proximal end of the first section may be secured to a handle. The distal end of the second section may further comprise a seal.

In another embodiment of the present invention, at least the second section of the core element may be composed of a heat-sensitive shape memory polymer that straightens in cold liquid and resumes a preformed configuration in hot liquid.

In accordance with an aspect of the present disclosure, a medical device may include: a core element having a first section extending substantially linearly, a second section capable of transitioning between an expanded configuration and a substantially linear configuration, and a longitudinal lumen having a first portion disposed in the first section and a second portion disposed in the second section; and an elongated member disposed within the lumen of the core element and moveable within the first and second portions of the lumen relative to the core element.

Various embodiments of the invention may include one or more of the following aspects: the core element may comprise a shape memory material; the expanded configuration may be a helical coil; the first section may comprise a proximal portion of the core element; the second section may comprise a distal portion of the core element; the elongated member may be extendable into and retractable out from the second portion of the longitudinal lumen; the second section of the core element may be in an expanded configuration when the elongated member is retracted from the second portion of the lumen; the second section of the core element may be in a substantially linear configuration when the elongated member is extended into the second portion of the lumen; the device may further comprise a handle operably connected to the core element and the elongated member; the core element may comprise a substantially constant outer diameter throughout the first and second; the first section of the core element may comprise a first outer diameter and the second section of the core element may comprise a second outer diameter different from the first outer diameter; the first section and second sections of the core element may comprise the same material; the first section of the core element may comprise a first material and the second section of the core element may comprise a second material different from the first material; the second material may be stiffer than the first material; the lumen may be defined by an imperforate wall; the second section may comprise a laser resistant material; the elongated member may include a wire.

In another aspect of the present disclosure, a medical device may include a core element having a first section extending substantially linearly, and a second section capable of transitioning between an expanded configuration and a substantially linear configuration, wherein the second section transitions between the expanded configuration and the substantially linear configuration in response to a change in temperature.

Various embodiments of the invention may include one or more of the following aspects: the core element may comprise a thermally actuated shape memory material; the expanded configuration may be a helical coil; the first section may comprise a proximal portion of the core element; the second section may comprise a distal portion of the core element; the second section of the core element may be in an expanded configuration when subjected to a medium having a temperature between approximately 95° F. and approximately 120° F.; the medium may comprise a liquid; the second section of the core element may be in a substantially linear configuration when subjected to a medium having a temperature between approximately 70° F. and approximately 100° F.; the medium may comprise a liquid; the core element may comprise a substantially constant outer diameter throughout the first and second sections; the first section of the core element may comprise a first outer diameter and the second section may comprise a second outer diameter different from the first outer diameter; the first section and second section of the core element may comprise the same material; the first section of the core element may comprise a first material and the second section of the core element may comprise a second material different from the first material; the second material may be stiffer than the first material.

In yet another aspect of the present disclosure, a device for manipulating material in the body of a patient may include a core element comprising a laser resistant material; a first section extending substantially longitudinally; a second section being wound to form a helical coil; a lumen extending longitudinally from at least a portion of the first section to at least a portion of the second section; and an imperforate wall defining the lumen, wherein the thickness of the imperforate wall is at least one half the diameter of the lumen.

Various embodiments of the invention may include one or more of the following aspects: the ratio of the thickness of the imperforate wall to the diameter of the lumen may be 0.8 to 1.0; at least the second section may comprise a laser resistant material; the laser resistant material may further comprise a shape memory material; the laser resistant shape-memory material may comprise a polymeric material; the polymeric material may further comprise a heat shrink extrusion; the polymeric material is selected from a group comprising polytetrafluoroethylene, expanded polytetrafluoroethylene, ethylene-tetrafluoroethylene, ethylene vinyl acetate, polyethylene, high-density polyethylene, medium-density polyethylene, polyurethane, nylon, polyvinyl chloride, polyimide and polyetherether ketone; the device may further comprise an elongated member axially positioned and slideably moveable within the lumen of the core element; helical coil may taper in diameter from a larger diameter at the proximal end of the second section to a smaller diameter at the distal end of the second section; the proximal end of the first section may be secured to a handle; the distal end of the second section may comprise a seal.

In a further aspect of the present disclosure, a device for manipulating material in the body of a patient may include a core element comprising a first section extending substantially longitudinally and a second section being wound to form a helical coil, wherein at least the second section comprises a shape-memory polymer, the shape-memory polymer capable of straightening in cold liquid and resuming a preformed configuration in hot liquid.

Various embodiments of the invention may include a helical coil that tapers in diameter from a larger diameter at the proximal end of the second section to a smaller diameter at the distal end of the second section.

Another aspect of the present disclosure includes a method of entrapping an object within a body. The method includes inserting a medical device into a body lumen, the medical device having a core element having a first section extending substantially linearly, a second section capable of transitioning between an expanded configuration and a substantially linear configuration, and a longitudinal lumen having a first portion disposed in the first section and a second portion disposed in the second section, and an elongated member disposed within the lumen of the core element and moveable within the first and second portions of the lumen relative to the core element. The method further includes advancing the medical device to a desired body location within the body lumen, transitioning the second section from the substantially linear configuration to the expanded configuration, and entrapping the object with the second section in the expanded configuration.

Various embodiments of the invention may include one or more of the following aspects: the method may further comprise the step of removing the medical device and entrapped object from the body; the core element may comprise a shape memory material; the step of transitioning the second section from the substantially linear configuration to the expanded configuration may include retracting the elongated member proximally out of the second portion of the lumen; the expanded configuration may be a helical coil; the method may further comprise the step of transitioning the second section from the expanded configuration to the substantially linear configuration; the step of transitioning the second section from the expanded configuration to the substantially linear configuration may include extending the elongated member into the second portion of the lumen; the step of transitioning the second section from the expanded configuration to the substantially linear configuration may release the entrapped object from the device; the core element may comprise a substantially constant outer diameter throughout the first and second sections; the first section of the core element may comprise a first outer diameter and the second section of the core element may comprise a second outer diameter different from the first outer diameter; the elongated member may include a wire.

A further aspect of the present disclosure includes a method of entrapping an object within a body. The method includes inserting a medical device into a body lumen, the medical device having a core element having a first section extending substantially linearly, and a second section capable of transitioning between an expanded configuration and a substantially linear configuration. The method further includes advancing the medical device to a desired body location within the body lumen, transitioning the second section from the substantially linear configuration to the expanded configuration in response to a change in temperature of the second section, and entrapping the object with the second section in the expanded configuration.

Various embodiments of the invention may include one or more of the following aspects: the method may further comprise the step of removing the medical device and entrapped object from the body; the core element may comprise a thermally actuated shape memory material; the step of transitioning the second section from the substantially linear configuration to the expanded configuration may include exposing the second section of the core element to a medium having a temperature between approximately 95° F. and approximately 120° F.; the medium may comprise a liquid; the expanded configuration may be a helical coil; the method may further comprise the step of transitioning the second section of the core element from the expanded configuration to the substantially linear configuration; the step of transitioning the second section of the core element from the expanded configuration to the substantially linear configuration may include exposing the second section of the core element to a medium having a temperature between approximately 70° F. and approximately 100° F.; the medium may comprise a liquid; the step of transitioning the second section from the expanded configuration to the substantially linear configuration may release the entrapped object from the device; the core element may comprise a substantially constant outer diameter throughout the first and second sections; the first section of the core element may comprise a first outer diameter and the second section of the core element comprises a second outer diameter different from the first outer diameter.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the invention and together with the description, serve to explain the principles of the invention. In the drawings, like reference characters generally refer to the same parts throughout the different views. Also, the drawings are not necessarily to scale, emphasis instead being placed upon illustrating the principles of the invention.

FIG. 1 is a schematic view of a medical device according to an illustrative embodiment of the invention.

FIG. 2 is a cross-sectional view of the medical device illustrated in FIG. 1 taken at line C-C.

FIG. 3A is a schematic view of the medical device illustrated in FIG. 1 with an elongated member inserted within a portion of the lumen that extends through the second section of the core element, according to another illustrative embodiment of the invention.

FIG. 3B is a schematic view of a medical device according to an illustrative embodiment of the invention.

FIG. 4 is a schematic view of a portion of a medical device according to an illustrative embodiment of the invention.

FIG. 5 is a schematic view of a portion of the medical device illustrated in FIG. 4, including the second section in a helical coil configuration according to an illustrative embodiment of the invention.

FIG. 6 is a schematic view of a portion of the medical device illustrated in FIG. 5, including a core element in a substantially linear configuration according to an illustrative embodiment of the invention.

### DESCRIPTION OF THE EMBODIMENTS

Reference will now be made in detail to the present exemplary embodiments of the invention, examples of which are illustrated in the accompanying drawings. Wherever possible, the same reference numbers will be used throughout the drawings to refer to the same or like parts. As used in this disclosure, “distal” refers to a position or direction furthest from a user of the device and “proximal” refers to a position or direction opposite distal and closest to the user.

Embodiments of the invention relate generally to medical devices for entrapping and/or removing material from within an anatomical lumen of a patient. Exemplary embodiments of the invention are generally applicable to any anatomical lumen, and may be used to remove material present in the urinary and/or biliary tracts of an anatomical lumen. According to exemplary embodiments, the invention may be useful in removing small concretions from within a body tract, for example, concretions that have fragmented during a lithotripsy procedure. Generally, an embodiment of a medical device according to the present invention includes a portion capable of transitioning between an expanded configuration and a substantially linear configuration without the use of a sheath. Medical devices according to embodiments of the present invention may have any desired shape, configuration, and/or dimensions suitable for deployment within a desired body lumen.

In some embodiments, medical devices in accordance with the present invention may have an overall length between approximately 50 cm and approximately 250 cm. In other embodiments, the devices may comprise a total length between approximately 140 cm and 200 cm, and preferably about 200 cm. However, these dimensions are exemplary and the overall length and diameter of the medical devices may vary depending on the application. Similarly, medical devices in accordance with the present invention may be utilized in treating animals, and thus be configured to be adaptable to varying anatomies.

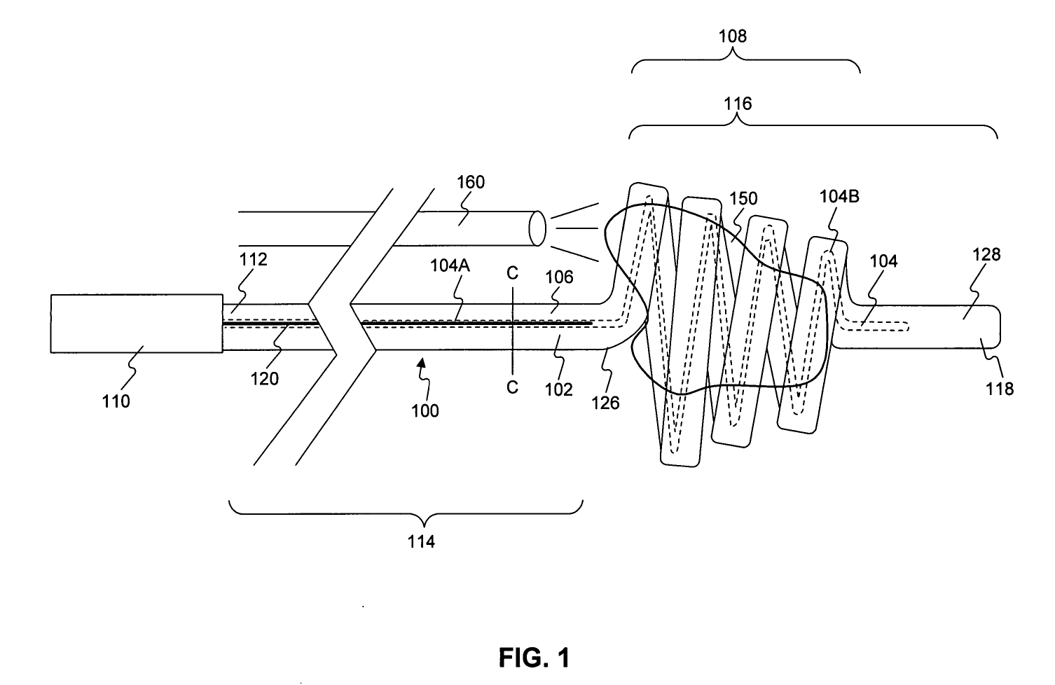

FIGS. 1-6 depict certain configurations of exemplary embodiments of medical devices 100 and 200 in accordance with the present invention. In general, all of the following embodiments of the medical device include a core element having at least a portion capable of transitioning between an expanded configuration and a substantially linear configuration. In accordance with one embodiment of the present invention and as shown in FIGS. 1-3B, the medical device 100 includes a core element 102 having a proximal first section 114 and a distal second section 116. The first section 114 extends substantially linearly and includes a proximal end 112 that may be secured to a suitable handle 110. The second section 116 extends distally from first section 114, may comprise a seal 118, and may transition between an expanded configuration and a substantially linear configuration.

Core element 102 may be fabricated from any known process such as, for example, extrusion. Core element 102 may have any desired cross-sectional shape and/or configuration. For example, core element 102 may have a substantially circular cross-section. Core element 102 may also have one or more cross-sectional shapes and/or configurations along its length, and may be any desired dimension suitable for deployment within a desired body lumen. For example, core element 102 may have dimensions adapted for placement in a biliary duct having a particular size. For instance, core element 102 may have an outer periphery that is substantially the same size as, or a size less than, the inner periphery of a biliary duct. The overall length and diameter of core element 102 may vary depending on the application. For example, a relatively long core element 102 may be advantageous for retrieving stones or other concretions deep within the body of a patient. Furthermore, core element 102 may also be flexible along at least a portion of its length so that it may bend as it is advanced through tortuous body lumens.

Core element 102 may be made from any suitable laser resistant shape memory material. Shape memory material can be formed into a particular shape, retain that shape during resting conditions (e.g., when the shaped material is in free space or when external forces applied to the shaped material are insufficient to substantially deform the shape), be deformed into a second shape upon subjecting the initial shape to a sufficiently strong external force, and revert substantially back to the initial shape once the external force is no longer applied, as is known in the art. Examples of shape memory materials include, but are not limited to, synthetic plastics (e.g., polytetrafluoroethylene (PTFE), expanded polytetrafluoroethylene (ePTFE), ethylenetetrafluoroethylene (ETFE), ethylene vinyl acetate (EVA), polyethylene (PE), high-density polyethylene (HDPE), medium-density polyethylene (MDPE), polyurethane, nylon, polyvinyl chloride (PVC), polyimide, polyetherether ketone (PEEK), and any other suitable polymeric material comprising heat shrink extrusion), stainless steel, and superelastic metallic alloys of nickel/titanium (e.g., nitinol), copper, cobalt, vanadium, chromium, iron, or the like.

In addition, any other suitable material exhibiting laser resistant characteristics such as, for example, a light color capable of reflecting most of the laser energy used during a lithotripsy procedure and absorbing or dissipating the energy not reflected with no or minimal damage to core element 102 at normal laser operating levels may also be used. Additional suitable materials will be apparent to those skilled in the art.

As seen in FIG. 1, the second section 116 may be configured such that it may transition between an expanded configuration and a substantially linear configuration. For example, the second section 116 may wind to form helical coil 108 in the absence of any additional forces and/or when core element 102 is in a relaxed or unstressed state. In addition, helical coil 108 may be configured such that it may selectively unwind and temporarily assume a substantially linear configuration when subjected to stresses or forces, for example, along a longitudinal axis of core element 102. Helical coil 108 may have any desired shape and/or configuration, and any desired dimensions suitable for deployment in a desired body lumen. For example, the second section 116 may be wound in a manner such that helical 108 is configured to taper from a larger diameter at the proximal end 126 of the second section 116 to a smaller diameter at the distal end 128 of the second section 116. Alternatively, helical coil 108 may include a larger diameter at the distal end 128 and a smaller diameter at the proximal end 126. In addition, helical coil 108 may have dimensions adapted for placement in a desired body lumen having a particular size. For instance, helical coil 108 may have a maximum outside diameter that is substantially the same size as, or slightly greater than, the inside diameter of a desired body lumen. Helical coil 108 in such configurations may be adapted to ensnare objects of various sizes from within an anatomical lumen, for example, a kidney stone from within the ureter of a patient.

The length, number of turns, maximum diameter, and pitch of helical coil 108 may vary depending on the application. In some embodiments, helical coil 108 may have between approximately 3 turns and approximately 10 turns, and preferably approximately 5 to approximately 7 turns. In another embodiment, the proximal end of the helical coil 108 may have a maximum diameter between approximately 0.2 cm and approximately 3.0 cm, and preferably approximately 0.7 cm to approximately 1.0 cm. In yet another embodiment, the overall length of the helical coil 108 may be between approximately 0.5 cm and approximately 3.0 cm, and preferably approximately 1.5 cm. In a further embodiment, the pitch of helical coil 108 may be such that adjacent turns may be in abutting relation with each other or be separated by small gaps that may be approximately 2 mm wide. These dimensions are exemplary and other suitable dimensions depending, for example, on the desired application, may be used.

Helical coil 108 may be formed in a number of different ways. For example, helical coil 108 may be formed by wrapping a portion of core element 102 around a mandrel to form the desired coil-like configuration. Core element 102 may then be subjected to heat treatment at a pre-determined temperature for a pre-determined length of time such that core-element 102 retains the coil-like shape when removed from the mandrel and the heat. As is known to those skilled in the art, the portion of core element 102 that has been heat treated to retain the desired coil-like configuration is capable of returning to the set desired coil-like configuration even after drastic deformation such as, for example, straightening.

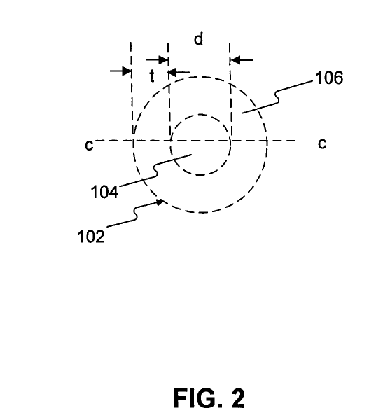

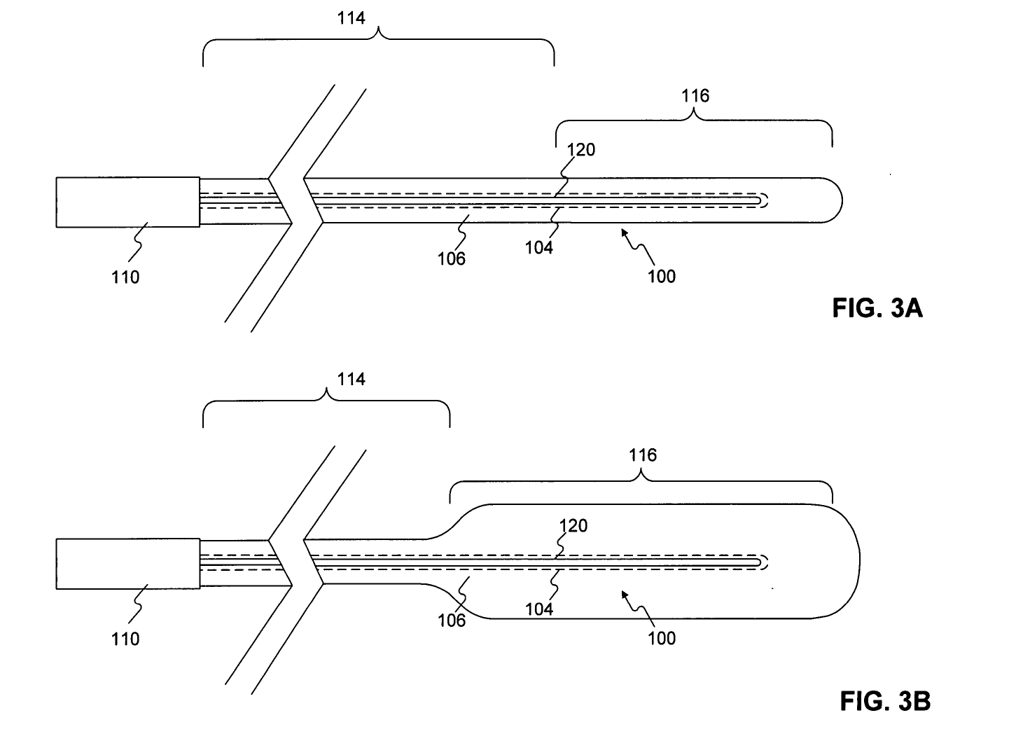

As shown in FIGS. 2 and 3A, some embodiments of the medical device 100 may comprise a core element 102 having a substantially constant outer diameter, a substantially constant inner diameter d, and a substantially constant wall thickness t throughout the first section 114 and the second section 116. Alternatively, and as shown in FIG. 3B, the second section 116 of core element 102 may comprise a larger outer diameter (e.g., cross-sectional area) than the first section 114. In keeping the inner diameter d of lumen 104 constant throughout the first section 114 and second section 116, the thickness t of wall 106 throughout the second section 116 may then be made greater than that of the first section 114. Increasing the wall thickness t for the second section 116, while maintaining a reduced wall thickness t for the first section, may increase the stiffness of the helical coil 108, while simultaneously maximizing the space available in an endoscope's working channel. A stiffer helical coil 108 will facilitate the use of the device as a backstop during lithotripsy, and as a sweeping device for removal of stones from the ureter. Maximizing space in the endoscope's working channel will increase irrigation to the site and visibility to the operator.

As an alternative or in addition to altering the diameter of portions of core element 102, stiffer materials may be used to achieve greater coil stiffness without compromising the space available in an endoscope's working channel. For example, the entire core element 102 may comprise a constant outer diameter, with the second section 116 made from a material stiffer (e.g., a greater durometer) than the material used for the first section 114. This may be accomplished, for example, by using multiple extruded tubes of varying stiffness, or a single extruded tube having varying stiffness.

Core element 102 may further include at least one longitudinally disposed lumen 104 defined by an imperforate wall 106. Alternatively, wall 106 may be perforated. Lumen 104 may have a first portion 104A disposed in the first section 114 and a second portion 104B disposed in the second section 116. In certain embodiments, lumen 104 may extend throughout the first section 114 and the second section 116. Lumen 104 may be formed by any suitable process such as, for example, extrusion, and may have any suitable cross-sectional shape, dimension, and/or configuration. For example, in some embodiments, lumen 104 may have a substantially circular cross-section. Alternatively, core element 102 may include a plurality of lumens (not shown) that may or may not differ in size or configuration. In such embodiments, the plurality of lumens may provide passageways useful in delivering, or removing, for example, medical devices, fluids, and/or other structures to or from a treatment site.

According to an embodiment of the present invention, FIG. 2 is a diagram illustrating exemplary dimensional ratios of a cross-section of the medical device 100 illustrated in FIG. 1. In one embodiment, a wall 106 may have a thickness t that is at least one-half the diameter d of lumen 104. The ratio of the thickness t of the wall 106 to the diameter d of lumen 104 may be between 0.5:1 and 2:1. In certain embodiments, the preferred ratio of the thickness t of wall 106 to the diameter d of lumen 104 may be 0.8:1.0. These dimensional ratios are exemplary and other suitable dimensional ratios depending, for example, on the desired application, may be used.

In some embodiments of the medical device 100, helical coil 108 may be configured to deform into a substantially linear configuration when subjected to additional forces or stresses. For example, an elongated member 120, may be axially positioned and slideably moveable with lumen 104 of core element 102, and may be configured to temporarily deform helical coil 108 into a substantially linear configuration without significantly affecting the ability of core element 102 to revert back into the coil-like configuration. For instance, as shown in FIGS. 3A and 3B, elongated member 120 may be configured to exert a force large enough to reversibly deform helical coil 108 into a substantially linear configuration when advanced distally through into the second portion 104B of lumen 104 disposed in the second section 116. In addition, elongated member 120 may be configured to be retracted proximally out of the second portion 104B of lumen 104, and out of the second section 116 such that core element 102 may again wind to form helical coil 108. In such embodiments, the medical device 100 may be deformed into a substantially linear configuration by elongated member 120 to facilitate placement at a desired body location within a desired body lumen. Once the medical device 100 is positioned at the desired body location, elongated member 120 may be retracted proximally out of the second portion 104B of lumen 104 that extends through the second section 116 to allow second section 116 to re-wind and again form helical coil 108.

Elongated member 120 may be advanced distally into the second portion 104B of lumen 104 and retracted proximally out of the second portion 104B of lumen 104 using any method known in the art. For example, any suitable actuator (not shown) in handle 110 may be used by a user. Elongated member 120 may be made from any suitable material such as, for example, wire. Additionally, elongated member 120 may have any suitable cross-sectional shape, dimension, configuration, and may be made from any suitable material that provides sufficient strength and flexibility for adequate operation.

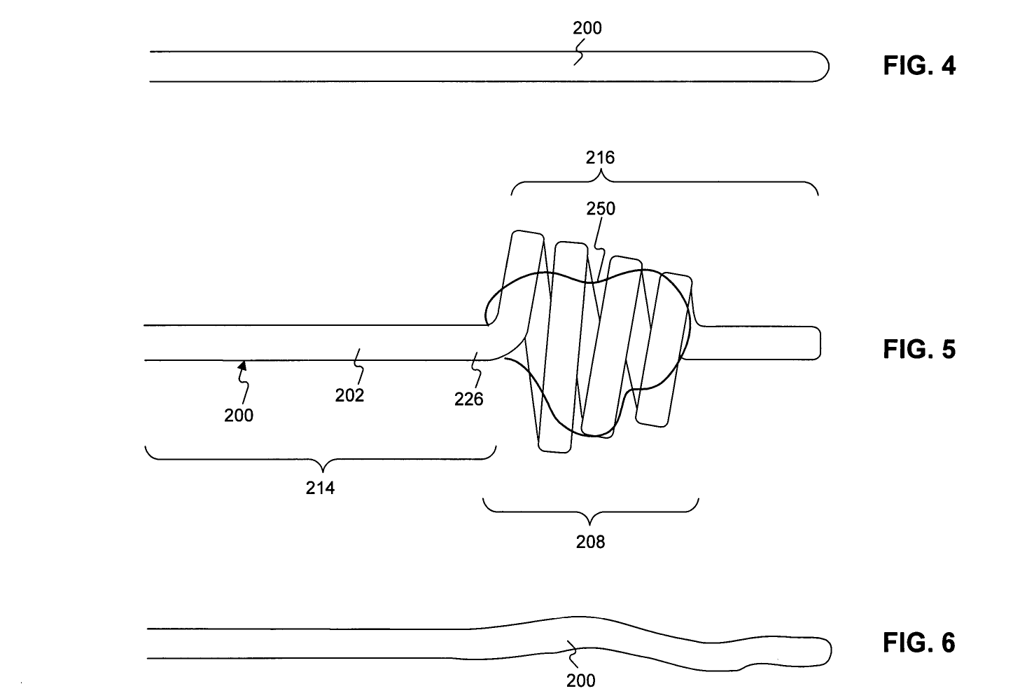

Alternatively, and as shown in FIGS. 4-6, the medical device 200, in accordance with one embodiment of the present invention, may include a core element 202 having a proximal first section 214 that may extend substantially linearly and a distal second section 216 that may transition between an expanded configuration and a substantially linear configuration. For example, the second section 216 may be wound to form helical coil 208 in accordance with aspects of the present invention. In such embodiments, at least the second section 216 may be constructed from a thermally actuated shape memory polymer that is capable of achieving a relaxed state and/or a substantially linear configuration in a cold medium having a temperature between approximately 70° F. and approximately 100° F., and reverting to a pre-formed expanded configuration such as, for example, a helical coil in a hot medium having a temperature between approximately 95° F. and approximately 120° F. Core element 202 may be made from any suitable material. Such materials may include, but are not limited to, Vestenamer™ (Degussa Corporation, Parsippany, N.J.) and Norsorex® (Atochem North America, Inc., Philadelphia, Pa.).

Core element 202 may or may not include longitudinally disposed lumens. In embodiments where longitudinally disposed lumens are provided in core element 202, the lumens may provide passageways useful in delivering, or removing, for example, medical devices, fluids, and/or other structures to or from a treatment site.

In accordance with aspects of a previously discussed embodiment, core element 202 may or may not comprise a substantially constant outer diameter throughout the first section 214 and the second section 116. For example, the second section 216 may comprise a larger outer diameter (e.g., cross-sectional area) than the first section 214. Furthermore, as an alternative or in addition to altering the diameter of portions of core element 202, stiffer materials may be used to achieve greater coil stiffness without compromising the space available in an endoscope's working channel. For example, the entire core element 202 may comprise a constant outer diameter, with the second section 216 made from a material stiffer (e.g., a greater durometer) than the material used for the first section 214.

Helical coils 108 and 208, shown and described in FIGS. 1 and 5, are an exemplary configuration of core elements 102 and 202 that may be used in the method and device of the present invention. Any other configuration capable of entrapping, releasing, and/or removing objects from within a body lumen may be used.

In reference to FIGS. 1-6, embodiments of the invention may include methods of using the medical devices 100 or 200 of the present disclosure to retrieve stones and other unwanted materials located in the bladder, ureter, kidney, or other body structures. Medical devices 100 or 200 may be used in an environment that is relatively fluid filled or that is relatively dry. The medical devices 100 or 200 may be inserted through the urethra of a patient or, alternatively, the medical devices 100 or 200 may be inserted percutaneously. The medical devices 100 or 200 may be used in any location of the body in which a passageway or orifice includes unwanted material to be removed and/or is being at least partially blocked.

Medical devices 100 or 200 may be advanced to a treatment site in a number of different ways. For example, some embodiments of medical devices 100 or 200 may include a suitable guide wire lumen (not shown) for advancing the devices 100 or 200 to a desired body location over a guide wire (not shown), wherein the desired body location may correspond to the vicinity of a stone or another object targeted for removal. Medical devices 100 or 200 may also be advanced to the treatment site through an access sheath or any other access device known in the art.

Alternatively, the medical devices 100 or 200 may be fed to the desired body location by means of an imaging device, and may travel through the body without the use of a guide wire or access sheath. To facilitate such feeding, core elements 102 and 202 may include radiopaque marker bands (not shown) detectable by x-ray or other imaging means. Thus, the user may monitor the position of the devices 100 or 200 and movement thereof through the use of an imaging device.

As yet another alternative, the medical devices 100 or 200 may be used in conjunction with an endoscope (not shown) or any other type of intracorporeal scope known in the art. The endoscope may travel through the body to the treatment site in any conventional manner. Once the endoscope is positioned adjacent to the treatment site, the medical devices 100 or 200 may be fed through an access port in the endoscopic sheath to gain access to the object targeted for entrapment, such as, for example, a stone.

The object targeted for entrapment may be a bodily concretion such as, for example, a stone 150. The object targeted for entrapment may include, but is not limited to, kidney stones, gallbladder stones, uric acid stones, or other solids commonly removed from a body structure or passageway. The objects may be of any size and/or shape. Furthermore, medical devices 100 or 200 may be used to entrap and/or retrieve objects that are both impacted and free floating.

In accordance with one embodiment of the present invention and in clinical applications where an endoscope (not shown) or other similar access device is used for access, the endoscope may be advanced through a tortuous body lumen and past a desired body location. With the second section 116 in a substantially linear configuration, the medical device 100 may then be inserted into the body lumen (not shown) by feeding the medical device 100 through an access port in the endoscopic sheath to the desired body location. For example, the medical device 100 may be advanced until the proximal end 126 of the second section 116 has a reached a desired location, such as, a short distance past an object targeted for entrapment and/or removal. The method is not limited to the use with any particular object, and may also be used with one or more intracorporeal and/or extracorporeal objects at various locations and of various geometries and compositions.

Once the proximal end 126 of the second section 116 is advanced past a targeted object, helical coil 108 may be deployed by retracting elongated member 120 proximally from the second portion 104B of lumen 104 disposed within the second section 116. As discussed above, any suitable actuator assemblies may be used to effect retraction of elongated member 120 and deployment of helical coil 108. When fully deployed, helical coil 108 may be substantially conical in shape or have any other configuration suitable to the particular size and shape of both the targeted object and/or environment it is located in. The proximal end 126 may or may not be the widest portion of the helical coil 108.

Once the helical coil 108 has been fully deployed, it may be moved proximally such that it sweeps the lumen and entraps the targeted object such as, for example, stone 150.

The medical device 100, along with the entrapped stone 150, may then be moved to any desired location, or may be allowed to remain stationary. For example, the medical device 100 may be used as an extraction device and removed from the body, assist in immobilizing objects and used as a backstop, or moved to a location more conducive to any additional procedures, such as lithotripsy with laser 160, surgery, or any other processes known in the art.

In certain circumstances, it may be desired to release the stone 150 after it has been captured in the helical coil 108. To do so, the helical coil 108 may be substantially straightened to release the entrapped stone 150. For example, this may be accomplished by distally advancing elongated member 120 into the second portion 104B of lumen 104 and into the second section 116 in order to deform the helical coil into a substantially linear configuration, and retracing the medical device 100 in the proximal direction, allowing the entrapped stone 150 to be released.

If desired, elongated member 120 may again be withdrawn from the second portion 104B of lumen 104 so that the second section 116 may revert back to the coil-like configuration, such that another targeted object may become entrapped within helical coil 108. Furthermore, the medical device 1 may also be re-used with the same patient, for example, by extending elongated member 120 distally into to the second portion 104B of lumen 104 and deforming helical coil 108 into a substantially linear configuration, and repositioning or reinserting the medical device 100 in the body.

In accordance with another embodiment of the present invention, medical device 200 may be inserted and positioned within a body lumen in a manner substantially similar to that used for medical device 100. For example, with the second section 216 in a substantially linear configuration, the medical device 200 may be inserted into the body lumen (not shown) by feeding the medical device 200 through an access port in the endoscopic sheath to the desired body location. The medical device 200 may be advanced until a proximal end 226 of the second section 216 has a reached a desired location, such as, a short distance past an object 250 targeted for entrapment and/or removal.

Once the proximal end 226 of the second section 216 is advanced past a targeted object 250, a medium having a temperature between approximately 95° F. and approximately 120° F. such as, for example, water, saline solution, or any other suitable liquid may be introduced through the endoscopic sheath to activate the thermally actuated core element 202 such that it deforms into a coil-like configuration such as, for example, helical coil 208. The medical device 200 may then be used in any suitable manner, examples of which are presented in this disclosure. To return the second section 216 to a substantially linear configuration, thermally actuated core element 202 may be exposed to a medium, for example, water, saline solution, or any other suitable liquid, having a temperature between approximately 70° F. and approximately 100° F. For example, this may be achieved by introducing cold water through the endoscopic sheath.

By way of example, one advantage of eliminating additional mechanisms, for example, a sheath surrounding core elements 102 and 202 to transition the second sections 116 and 216 of core elements 102 and 202 from one position to another is that the thickness of core elements 102 and 202 can be maximized until it reaches the internal diameter of a body tract. The increased thickness of core elements 102 and 202 may render the devices 100 and 200 more laser resistant and thus minimizes damage to core elements 102 and 202 caused by laser energy.

For exemplary purposes, various embodiments of the invention may be used to occlude anatomical tracts and lumens to prevent migration of foreign objects; sweep tracts or lumens to remove foreign objects; entrap foreign objects to move or remove the object; and/or substantially straighten the second sections 116 and 216 to release an entrapped object from helical coils 108 and 208.

Embodiments of the invention may be used in any medical or non-medical procedure, including any medical procedure where removal of an object from within a body lumen is desired. In addition, at least certain aspects of the aforementioned embodiments may be combined with other aspects of the embodiments, or removed, without departing from the scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A medical device comprising:
a core element having a first section extending substantially linearly, a second section capable of transitioning between an expanded configuration and a substantially linear configuration, and a longitudinal lumen having a first portion disposed in the first section and a second portion disposed in the second section; and
an elongated member disposed within the lumen of the core element and moveable within the first and second portions of the lumen relative to the core element.

2. The device of claim 1, wherein the core element comprises a shape memory material.

3. The device of claim 1, wherein the expanded configuration is a helical coil.

4. The device of claim 1, wherein the first section comprises a proximal portion of the core element.

5. The device of claim 1, wherein the second section comprises a distal portion of the core element.

6. The device of claim 1, wherein the elongated member is extendable into and retractable out from the second portion of the longitudinal lumen.

7. The device of claim 6, wherein the second section of the core element is in an expanded configuration when the elongated member is retracted from the second portion of the lumen.

8. The device of claim 6, wherein the second section of the core element is in a substantially linear configuration when the elongated member is extended into the second portion of the lumen.

9. The device of claim 1, further comprising a handle operably connected to the core element and the elongated member.

10. The device of claim 1, wherein the core element comprises a substantially constant outer diameter throughout the first and second sections.

11. The device of claim 1, wherein the first section of the core element comprises a first outer diameter and the second section of the core element comprises a second outer diameter different from the first outer diameter.

12. The device of claim 1, wherein the first section and second sections of the core element comprise the same material.

13. The device of claim 1, wherein the first section of the core element comprises a first material and the second section of the core element comprises a second material different from the first material.

14. The device of claim 13, wherein the second material is stiffer than the first material.

15. The device of claim 1, wherein the lumen is defined by an imperforate wall.

16. The device of claim 1, wherein the second section comprises a laser resistant material.

17. The device of claim 1, wherein the elongated member includes a wire.

18. A medical device comprising:
a core element having a first section extending substantially linearly, and a second section capable of transitioning between an expanded configuration and a substantially linear configuration, wherein the second section transitions between the expanded configuration and the substantially linear configuration in response to a change in temperature.

19. The device of claim 18, wherein the core element comprises a thermally actuated shape memory material.

20. The device of claim 18, wherein the expanded configuration is a helical coil.

21. The device of claim 18, wherein the first section comprises a proximal portion of the core element.

22. The device of claim 18, wherein the second section comprises a distal portion of the core element.

23. The device of claim 18, wherein the second section of the core element is in an expanded configuration when subjected to a medium having a temperature between approximately 95° F. and approximately 120° F.

24. The device of claim 23, wherein the medium comprises a liquid.

25. The device of claim 18, wherein the second section of the core element is in a substantially linear configuration when subjected to a medium having a temperature between approximately 70° F. and approximately 100° F.

26. The device of claim 25, wherein the medium comprises a liquid.

27. The device of claim 18, wherein the core element comprises a substantially constant outer diameter throughout the first and second sections.

28. The device of claim 18, wherein the first section of the core element comprises a first outer diameter and the second section comprises a second outer diameter different from the first outer diameter.

29. The device of claim 18, wherein the first section and second section of the core element comprise the same material.

30. The device of claim 18, wherein the first section of the core element comprises a first material and the second section of the core element comprises a second material different from the first material.

31. The device of claim 30, wherein the second material is stiffer than the first material.

32. A device for manipulating material in the body of a patient comprising:
a core element comprising
a laser resistant material;
a first section extending substantially longitudinally;
a second section being wound to form a helical coil;
a lumen extending longitudinally from at least a portion of said first section to at least a portion of said second section; and
an imperforate wall defining said lumen, wherein the thickness of said imperforate wall is at least one half the diameter of said lumen.

33. The device of claim 32, wherein the ratio of the thickness of said imperforate wall to the diameter of said lumen is 0.8 to 1.0.

34. The device of claim 32, wherein at least the second section comprises a laser resistant material.

35. The device of claim 34, wherein the laser resistant material further comprises a shape memory material.

36. The device of claim 35, wherein the laser resistant shape-memory material comprises a polymeric material.

37. The device of claim 36, wherein the polymeric material comprises a heat shrink extrusion.

38. The device of claim 36, wherein the polymeric material is selected from a group comprising polytetrafluoroethylene, expanded polytetrafluoroethylene, ethylenetetrafluoroethylene, ethylene vinyl acetate, polyethylene, high-density polyethylene, medium-density polyethylene, polyurethane, nylon, polyvinyl chloride, polyimide and polyetherether ketone.

39. The device of claim 32, further comprising an elongated member axially positioned and slideably moveable within said lumen of said core element.

40. The device of claim 32, wherein said helical coil tapers in diameter from a larger diameter at the proximal end of said second section to a smaller diameter at the distal end of said second section.

41. The device of claim 32, wherein the proximal end of said first section is secured to a handle.

42. The device of claim 32, wherein the distal end of said second section comprises a seal.

43. A device for manipulating material in the body of a patient comprising:
a core element comprising
a first section extending substantially longitudinally; and
a second section being wound to form a helical coil;
wherein at least the second section comprises a shape-memory polymer, the shape-memory polymer capable of straightening in cold liquid and resuming a preformed configuration in hot liquid.

44. The device of claim 43, wherein said helical coil tapers in diameter from a larger diameter at the proximal end of said second section to a smaller diameter at the distal end of said second section.

45. A method of entrapping an object within a body comprising:
inserting a medical device into a body lumen, the medical device comprising:
a core element having a first section extending substantially linearly, a second section capable of transitioning between an expanded configuration and a substantially linear configuration, and a longitudinal lumen having a first portion disposed in the first section and a second portion disposed in the second section; and
an elongated member disposed within the lumen of the core element and moveable within the first and second portions of the lumen relative to the core element;
advancing said medical device to a desired body location within the body lumen;
transitioning the second section from the substantially linear configuration to the expanded configuration; and
entrapping the object with the second section in the expanded configuration.

46. The method of claim 45, further comprising the step of removing the medical device and entrapped object from the body.

47. The method of claim 45, wherein the core element comprises a shape memory material.

48. The method of claim 45, wherein the step of transitioning the second section from the substantially linear configuration to the expanded configuration includes retracting the elongated member proximally out of the second portion of the lumen.

49. The method of claim 45, wherein the expanded configuration is a helical coil.

50. The method of claim 45, further comprising the step of transitioning the second section from the expanded configuration to the substantially linear configuration.

51. The method of claim 50, wherein the step of transitioning the second section from the expanded configuration to the substantially linear configuration includes extending the elongated member into the second portion of the lumen.

52. The method of claim 50, wherein the step of transitioning the second section from the expanded configuration to the substantially linear configuration releases the entrapped object from the device.

53. The method of claim 45, wherein the core element comprises a substantially constant outer diameter throughout the first and second sections.

54. The method of claim 45, wherein the first section of the core element comprises a first outer diameter and the second section of the core element comprises a second outer diameter different from the first outer diameter.

55. The method of claim 45, wherein the elongated member includes a wire.

56. A method of entrapping an object within a body comprising:
inserting a medical device into a body lumen, the medical device comprising:
a core element having a first section extending substantially linearly, and a second section capable of transitioning between an expanded configuration and a substantially linear configuration;
advancing the medical device to a desired body location within the body lumen;
transitioning the second section from the substantially linear configuration to the expanded configuration in response to a change in temperature of the second section; and
entrapping the object with the second section in the expanded configuration.

57. The method of claim 56, further comprising the step of removing the medical device and entrapped object from the body.

58. The method of claim 56, wherein the core element comprises a thermally actuated shape memory material.

59. The method of claim 56, wherein the step of transitioning the second section from the substantially linear configuration to the expanded configuration includes exposing the second section of the core element to a medium having a temperature between approximately 95° F. and approximately 120° F.

60. The method of claim 59, wherein the medium comprises a liquid.

61. The method of claim 56, wherein the expanded configuration is a helical coil.

62. The method of claim 56, further comprising the step of transitioning the second section of the core element from the expanded configuration to the substantially linear configuration.

63. The method of claim 62, wherein the step of transitioning the second section of the core element from the expanded configuration to the substantially linear configuration includes exposing the second section of the core element to a medium having a temperature between approximately 70° F. and approximately 100° F.

64. The method of claim 63, wherein the medium comprises a liquid.

65. The method of claim 56, wherein the step of transitioning the second section from the expanded configuration to the substantially linear configuration releases the entrapped object from the device.

66. The method of claim 56, wherein the core element comprises a substantially constant outer diameter throughout the first and second sections.

67. The method of claim 56, wherein the first section of the core element comprises a first outer diameter and the second section of the core element comprises a second outer diameter different from the first outer diameter.
